(19) United States
(12) Patent Application Publication
(10) Pub. No.: US 2009/0157192 A1
(43) Pub. Date: Jun. 18, 2009

(54) Replacement joint

(75) Inventor: J. Michael Stuart, Rio Rancho, NM (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: MicroDexterity Systems, Inc., Albuquerque, NM (US)

(21) Appl. No.: 12/002,008

(22) Filed: Dec. 14, 2007

(51) Int. Cl. A61F 2/32

(52) U.S. Cl. 623/22.15; 623/22.11

(57) ABSTRACT

A replacement joint for a human body and a method of installing such a replacement joint is provided. A ball portion of the replacement joint is installed on an end of a first bone. A receptacle having an engagement surface that has an aspherical configuration is formed in a second bone. A socket portion of the replacement joint is inserted in the receptacle so as to receive the ball portion on the first bone. The socket portion has an engagement surface with an aspherical configuration complementary to the engagement surface of the receptacle in the second bone. The configurations of the engagement surfaces of the socket portion and the receptacle in the second bone are such that inserting the socket portion in the receptacle in the second bone provides the socket portion with at least four degrees of constraint.

### BACKGROUND OF THE INVENTION

Implantation of a replacement joint is an increasingly common treatment for joint failures caused by injury or disease. One of the most commonly replaced joints is the hip. Known hip replacement joints include a large hemispherical acetabular component to replace the acetabulum in the pelvis and a femoral component having a head portion that is received in the acetabular component. The replacement procedure involves using a large bone reamer to create a hemispherical pocket in the pelvis into which the acetabular component is seated. The femoral component, in turn, is attached to the end of the femur. The replacement joint components are inserted through an incision in the patient's body. This incision often must be relatively large in order to accommodate the relatively large acetabular component. As is well known, a larger incision can lead to increased stress on the patient.

Because the pocket in the pelvis in which the acetabular component seats is hemispherical, the acetabular component is capable of rotating relative to the pelvis with two degrees of freedom before it is affixed in place. The acetabular component can be secured in place using cement or bone screws. In order to ensure proper fit, and hence operation, of the replacement joint, the acetabular and femoral components should be sized and oriented to match the bone structure of the patient. However, until it is secured in place, the acetabular component is capable of rotating relative to the pocket in the pelvis. Thus, getting the acetabular component into the proper alignment is very difficult and often the final alignment is merely an estimate or educated guess by the surgeon as to the proper position. Unfortunately, if the acetabular component is misaligned, problems can arise with the replacement joint including a limited range of motion or a joint dislocation.

### BRIEF SUMMARY OF THE INVENTION

The invention provides a replacement joint for a human body and a method of installing such a replacement joint. A ball portion of the replacement joint is installed on an end of a first bone. A receptacle having an engagement surface that has an aspherical configuration is formed in a second bone. A socket portion of the replacement joint is inserted in the receptacle so as to receive the ball portion on the first bone. The socket portion has an engagement surface with an aspherical configuration complementary to the engagement surface of the receptacle in the second bone. The configurations of the engagement surfaces of the socket portion and the receptacle in the second bone are such that inserting the socket portion in the receptacle in the second bone provides the socket portion with at least four degrees of constraint.

### BRIEF DESCRIPTION OF THE SEVERAL VIEWS OF THE DRAWING(S)

FIG. 1 is a perspective view showing the installation of a conventional prior art hemispherical acetabular socket in a complementary hemispherical pocket in a pelvis.

FIG. 2 is a schematic side view showing the conventional hemispherical acetabular socket of FIG. 1 installed in the pocket of FIG. 1.

FIG. 3 is a schematic partially cutaway side view of an illustrative embodiment of the replacement joint of the present invention in which the acetabular socket and mating receptacle in the bone have rectangular configurations.

FIG. 4 is a perspective view of an alternative embodiment of the present invention in which the receptacle in the bone for receiving the acetabular socket comprises a plurality of cavities.

FIG. 5 is enlarged perspective view showing the insertion of one of the plurality of elements of the acetabular socket being inserted in one of the cavities of the receptacle of FIG. 4.

FIG. 6 is a partially cutaway side view of the acetabular socket and receptacle of the embodiment of FIGS. 4 and 5 as installed in a pelvis with a ball on the end of the femur received in the acetabular socket.

FIG. 7 is an end view of the installed acetabular socket and receptacle of FIG. 6.

FIG. 8 is a cutaway side view of an alternative configuration for the cavities of the receptacle of the embodiment of FIGS. 4 and 5.

FIG. 9 is an end view showing the elements of the acetabular socket installed in the alternative cavities of the receptacle of FIG. 8.

FIG. 10 is a schematic block diagram of an illustrative robotic surgical system for use in installing the replacement joint of the present invention.

### DETAILED DESCRIPTION OF THE INVENTION

Referring now more particularly to FIGS. 3-6 of the drawings, there are shown exemplary embodiments of a replacement or prosthetic joint 10 for replacing a ball and socket type joint in the human body according to the present invention. The present invention is illustrated and described herein in the context of a replacement hip joint, however, those skilled in the art will appreciate that the invention is also applicable to other replacement ball and socket joints such as a replacement shoulder joint.

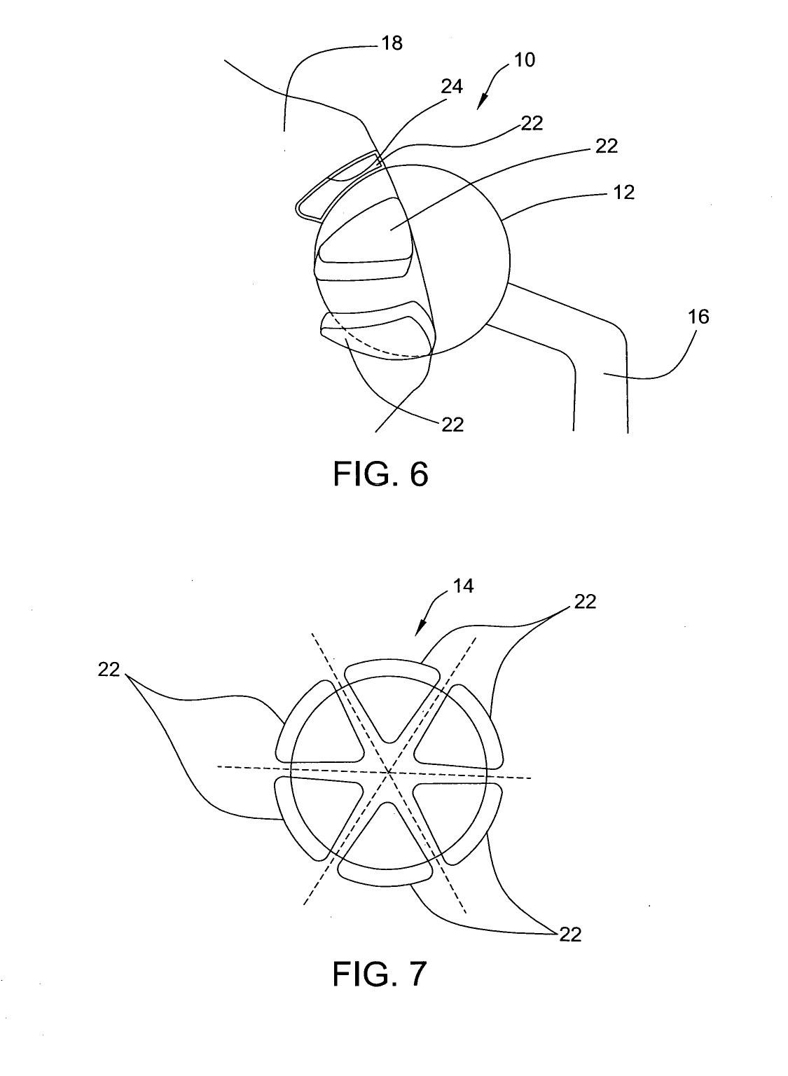

For a total hip replacement, the prosthetic joint includes both a ball portion 12 and an acetabular or socket portion 14. In this case, the ball portion 12 can include a stem or other connecting portion for connecting the ball portion 12 to the femur 16 and a generally spherical ball supported on the connecting portion (see e.g., FIGS. 3 and 6). When attached to the femur 16, the ball is arranged at the upper end of the femur. The ball portion 12 of the prosthetic joint 10 can be of conventional design and can be attached to the femur using known techniques.

For receiving the ball portion 12 at the end of the femur 16, the socket or acetabular portion or component 14 of the prosthetic joint 10 defines a generally hemispherical pocket or cup within which the ball portion 12 can move with three degrees of freedom. The acetabular socket component 14 is attached to the patient's pelvis 18 and in particular is mounted in a cavity or receptacle 20 that is formed in the pelvis 18. As will be appreciated from the following description, the acetabular socket component 14 of the present invention is equally applicable to total hip replacement procedures in which the end of the femur is replaced with a completely new ball portion as well as partial hip replacement procedures where a new prosthetic acetabular socket is installed and the entire original femur head is maintained or a only a portion of the original femur head is replaced.

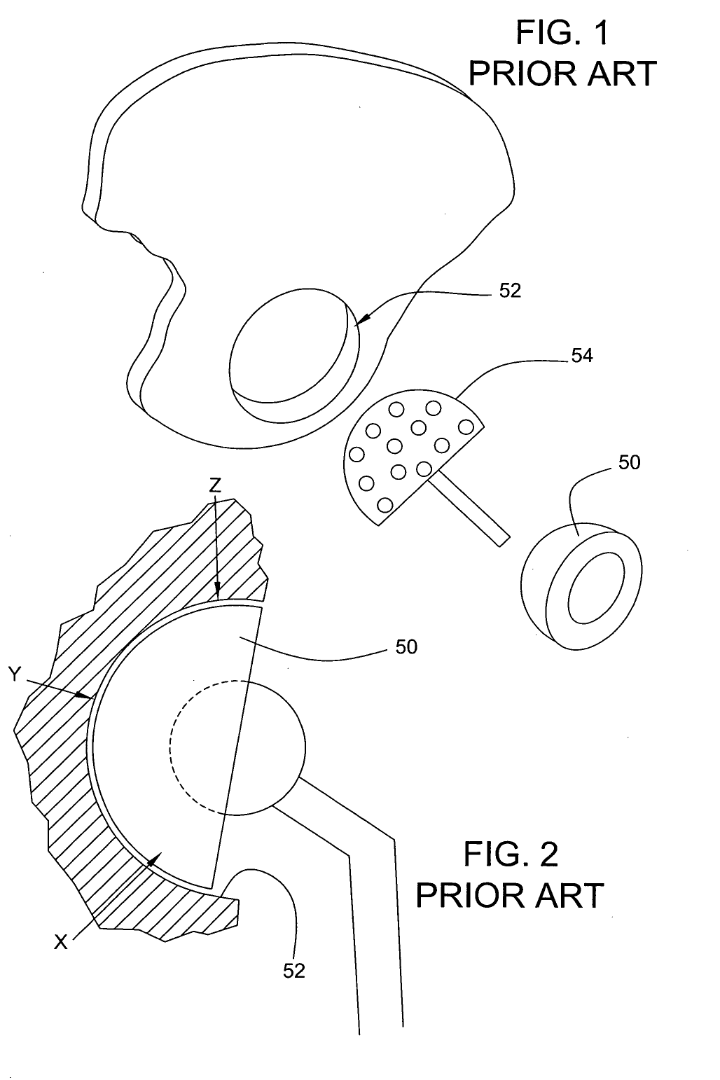

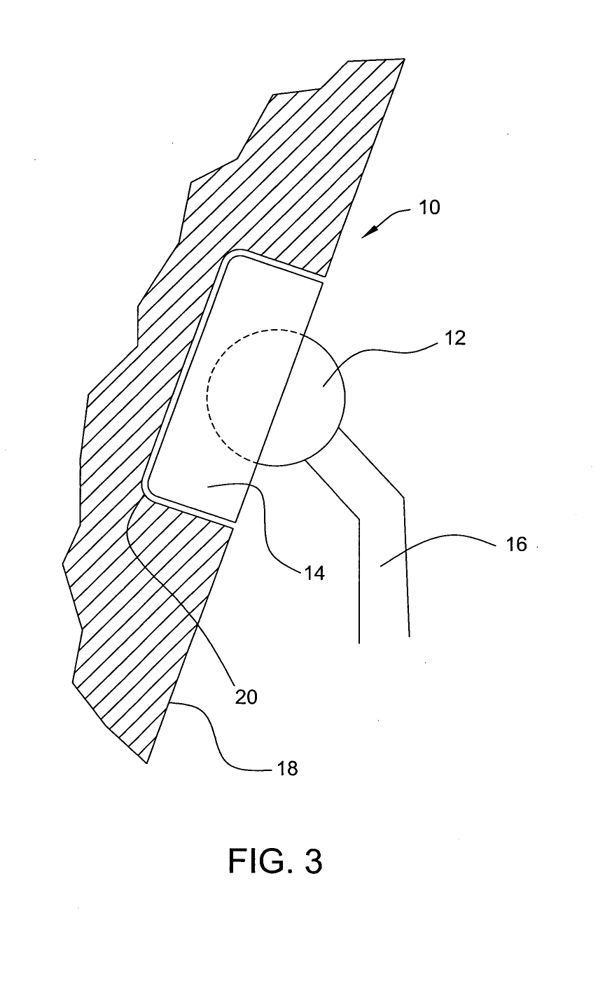

Current replacement acetabular sockets only have three degrees of constraint when they are placed in the mating cavity formed in the bone. In particular, as noted above and as shown in FIGS. 1 and 2, current replacement acetabular sockets 50 have a hemispherical configuration and mate with a complementary hemispherical cavity 52 in the pelvis formed by a bone reamer 54. Because of the mating hemispherical configurations, current acetabular sockets 50 can rotate relative to the cavity 52 formed in the pelvis until they are secured in place using cement or bone screws. This can make it quite difficult for surgeons to properly align and orient the acetabular socket 50.

According to the present invention, to enable a surgeon to more precisely align and orient the acetabular component relative to the patient's pelvis, the acetabular socket component 14 and the receptacle 20 in the patient's pelvis 18 are configured such that at least four degrees of constraint are provided when the acetabular socket component is inserted in the receptacle. This is accomplished by providing the acetabular socket component 14 and, in turn, the mating receptacle 20 formed in the pelvis with complementary aspherical configurations. More specifically, the acetabular socket component 14 and the mating receptacle 20 have engagement surfaces with complementary aspherical configurations (i.e., non-spherical or non-hemisherical) that interengage one another so that the receptacle 20 formed in the pelvis better grips and retains and the acetabular socket component 14 and in such manner that rotation of the actetabular socket component 14 relative to the pelvis 18 is prevented. As a result, a surgeon is able to position the acetabular socket component 14 in a highly precise manner, which should lead to a reduction in subsequent problems, including dislocations, with the replacement joint 10.

Any configuration that provides the necessary minimum of four degrees of constraint can be used for the engagement surfaces of the acetabular socket component 14 and the receptacle 20 in the pelvis. For example, according to one relatively simple embodiment shown in FIG. 3, the acetabular socket component 14 could have a body in the form of a rectangular cube with the cup for receiving the end of the ball portion 12 being formed in one side of the cube. The receptacle 20 in the pelvis 18 also has a rectangular configuration that is sized to receive the body of the acetabular socket component 14 with a relatively tight fit. The size of the body of the acetabular socket component 14 should be such that the complementary receptacle 20 will fit in the available space on the patient's pelvis and the size of the cup approximates the size of the original acetabulum in the pelvis. The unique, in this case, rectangular configurations of the acetabular socket component 14 and the mating receptacle 20 in the pelvis 18 prevent the rotation of the acetabular socket component relative to the pelvis that makes conventional socket designs difficult to orient precisely.

The acetabular socket component 14, as well as the ball portion 12, can be constructed of any suitable medically implantable materials such as metals, ceramics or plastics. Moreover, in a known manner, the ball and acetabular socket components 12, 14 could be provided with porous surfaces that would allow bone growth into the implant itself thereby helping improve retention of the replacement joint. The components of the replacement joint also could be provided with a surface coating that stimulates bone growth around the implant.

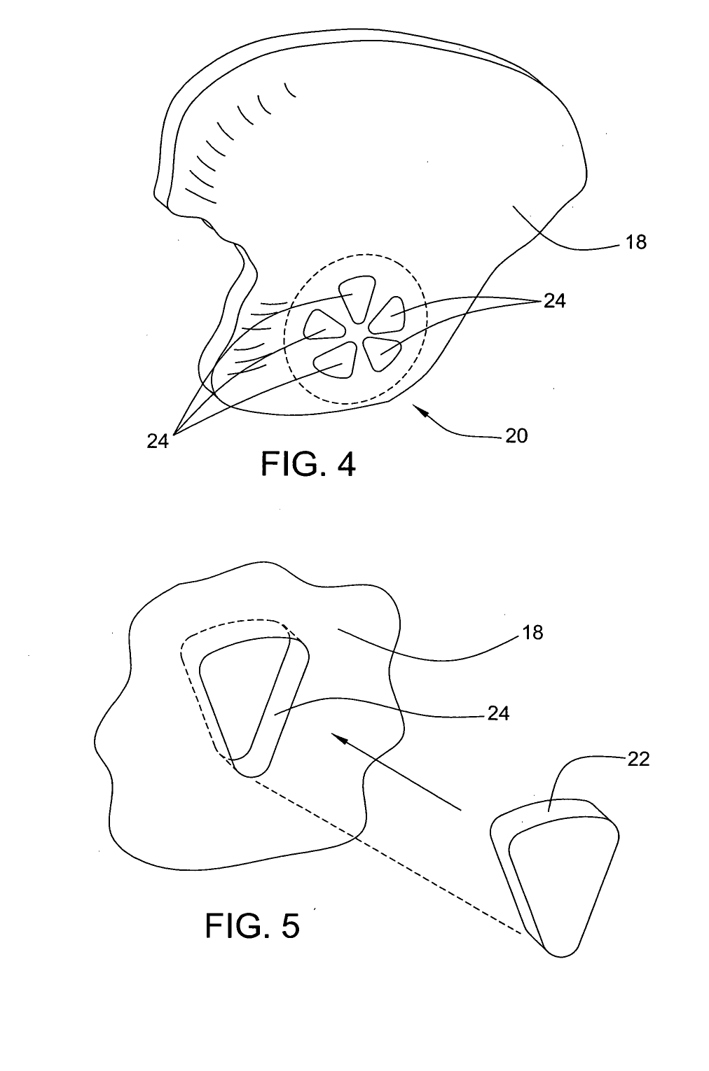

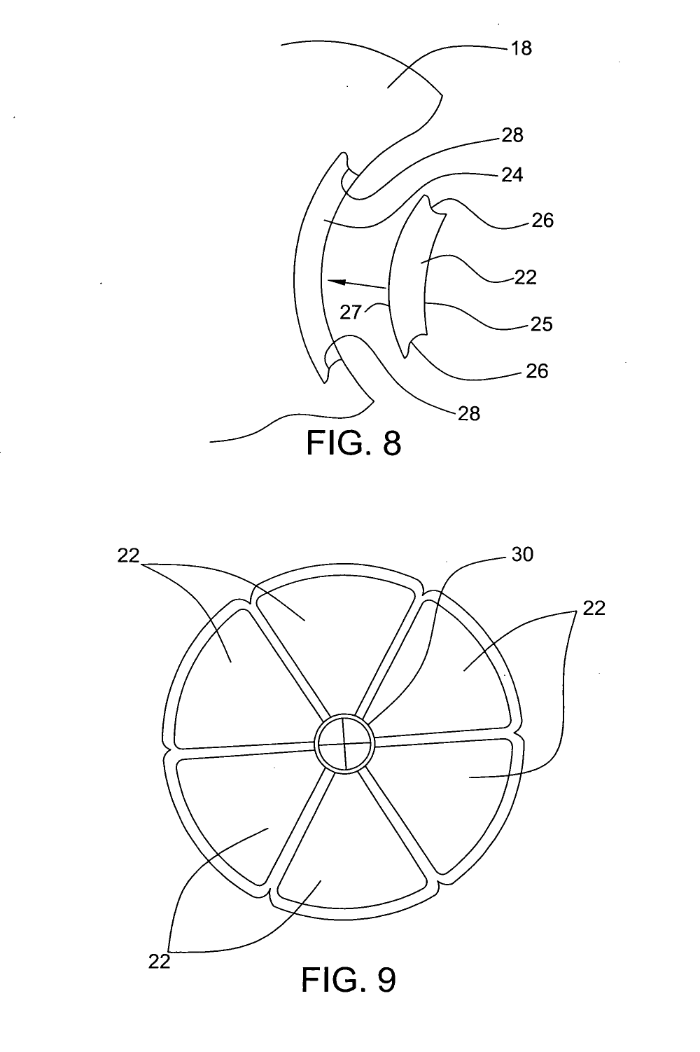

More complex shapes could also be used for the engagement surface of the acetabular socket component 14. Moreover, to allow the acetabular component 14 to be fed into the pelvis region of a patient using a smaller incision, thus making the hip joint replacement procedure less invasive, the component 14 could be divided into a plurality of smaller elements 22. In such a case, the receptacle 20 for the acetabular socket can also be divided into a plurality of cavities 24 each of which receives a respective element 22 of the acetabular socket. Again, in order to allow for precise placement and orientation of all of the elements of the acetabular socket component 14, each element 22 and each mating cavity 24 of the receptacle 20 can have complementary aspherical configurations that provide each element with at least four degrees of constraint when inserted into its respective cavity. An illustrative embodiment of such an arrangement is shown in FIGS. 4-7.

In the embodiment of FIGS. 4-7, the acetabular socket component 14 is divided into a plurality of wedge shaped elements 22 which together define the cup for receiving the head of the ball component 12. The receptacle 20 for receiving the socket component, in turn, comprises a plurality of wedge shaped cavities 24 with each wedge shaped element of the acetabular socket component being received in a corresponding one of the wedge shaped cavities. The corresponding cavity 24 for each element 22 of the acetabular socket 14 has a size and shape complementary to the size and shape of the respective socket element 22. As a result, the engagement surface of the socket element 22 engages the engagement surface of its respective cavity 24 when inserted therein providing the socket element 22 with at least four degrees of constraint. As with the simple rectangular configuration, the wedge shaped configurations of the socket elements 22 and the corresponding cavities 24 prevent the socket elements 22 from rotating once they are placed in their respective cavities. Thus, the invidual elements 22 of the acetabular socket 14 can be arranged in the precise desired positions relative to the pelvis 18 and femur 16 much more quickly and easily than current designs. In FIGS. 4-7, five to six substantially identically wedge shaped elements 22 are provided. Any number of elements 22 can be used so long as they provide the minimum of three contact points necessary to define the hemishperical cup for receiving the head of the femur.

The acetabular socket 14 of the present invention and any sub-elements 22 thereof can be secured in the receptacle 20 in the pelvis 18 using any suitable means including for example cement and/or one or more bone screws. Alternatively, one or more mechanical engagement features could be formed directly into the acetabular socket 14 and mating receptacle 20. For example, as shown in the embodiment illustrated in FIGS. 8-9, one or more of the outer edges 26 of each of the wedge elements 22 can be configured to taper radially outward as the edge 26 extends from the outer surface 25 of the element to the inner surface 27 of the element. The engagement surfaces defined by the mating edges 28 of each wedge-shaped cavity 24, in turn, have an undercut configuration with the edge 28 tapering outward as it extends downward from the outer surface of the bone. In this case, neither edge is tapered continuously or linearly and it will be appreciated that any number of suitable tapered or undercut configurations can be used. As shown in FIG. 9, a wedge screw 30 can then be inserted in the radial center of the array of wedge elements 22 in order to push the wedge elements radially outward so that the tapered edges 26 of the wedge elements 22 tightly engage the undercut edges 28 of the wedge shaped cavities 24 thereby locking the elements in place in the cavities. Thus, the combination of the wedge screw 20 and the tapered/undercut edges 26, 28 of the wedge elements 22 and the cavities 24 serve to secure the acetabular socket in the receptacle in the pelvis without the need for cement, although cement could also be used as desired.

The receptacle 20 and/or receptacle cavities 24 in the bone for receiving the one or more elements 22 of the acetabular socket 14 of the invention can be formed using conventional bone cutting tools. It is particularly advantageous if these bone cutting tools are supported and manipulated by medical robots or manipulators. As is known, such robots and manipulators can provide a number of advantages to both patients and medical practitioners. In particular, a robot or manipulator can enhance the dexterity of a surgeon/operator and even allow the surgeon to manipulate the tool in ways the surgeon would not be capable of achieving when using his own hands.

The robots or manipulators that can be used to help install the replacement joint 10 of the present invention can be master-slave controlled manipulators in which the surgeon inputs and/or movement signals to the “slave” manipulator via a master or haptic interface that operates through a controller or control console. Alternatively, the robot or manipulator can be a unit that is intended to be pre-programmed with the required tool movements before the surgical procedure thereby eliminating the need for a slave robot or manipulator. Of course, the robot or manipulator can be designed to operate using a combination of these two concepts with some of the required tool movements being pre-programmed and with the surgeon providing other positioning or movement signals during the actual procedure through a slave manipulator. Whether or not a haptic interface is provided, the manipulator would be under the control of a surgeon in some manner.

As will be appreciated, the one or more cavities 24 in the bone for receiving the one or more elements 22 of the acetabular socket component 14 have relatively complex shapes, particularly as compared to the bone reamer formed hemispherical cavities used to receive conventional replacement acetabular sockets. For example, the embodiment of the invention illustrated in FIGS. 8 and 9 uses wedge shaped cavities 24 with an undercut outer edge 28. While such a shape conceivably could be cut by hand by a surgeon, the advantage of the surgical robot or manipulator is that the necessary complex shapes of the cavities can be cut into the bone with minimal intervention to the patient. In essence, the robot or manipulator operates in manner akin to milling machine, enabling the more complex series of tool movements and cuts necessary to produce the cavities 22 to be made much more quickly. Examples of robots or manipulators suitable for use in installing the replacement socket of the present invention are disclosed in U.S. Pat. Nos. 6,676,669 and 6,723,106 and U.S. patent application Ser. No. 11/710,023 all of which are owned by the assignee of the present invention and which are incorporated herein by reference.

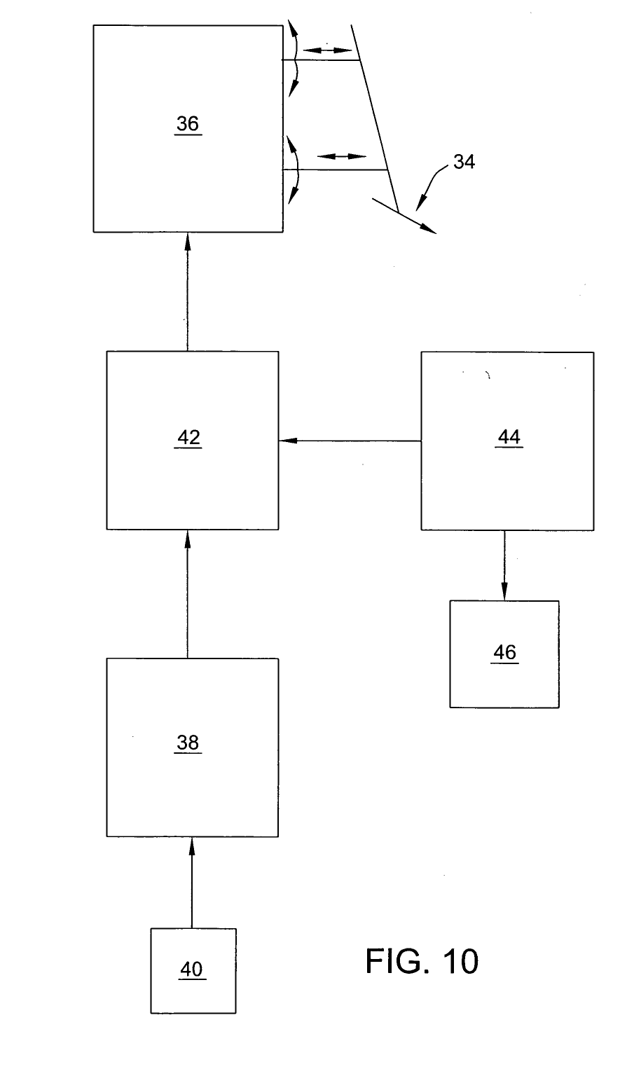

An illustrative embodiment of a robotic surgical system including a master-slave manipulator 36 that can be used installing the replacement joint 10 of the present invention and in particular cut the cavity or cavities 22 for receiving the acetabular socket component 14 is shown schematically in FIG. 10. The illustrated embodiment includes a manipulator 36 including arms that can support a tool, e.g. a bone cutting tool 34, and move it with at least one and preferably six degrees of freedom. The manipulator 36 can be of any suitable design including one of the designs disclosed in the aforementioned patents and application. The surgeon/operator provides movement input signals to the “slave” manipulator via a master or haptic interface 38 which operates through a controller or control console 42. Specifically, the manipulator 36 serves as a slave robot and the surgeon indicates the desired movement of the tool 34 held by the manipulator through the use of an input device 40 of the haptic interface 38 such as a six degree of freedom tool handle, joystick, foot pedal or the like. The haptic interface 38 relays these signals to the controller 42, which, in turn, applies various desired predetermined adjustments to the signals prior to relaying them to the slave manipulator 36. Any haptic interface can be used to control the manipulator 36 via the controller 42. Preferably, the haptic interface 38 has the same or more degrees of freedom than the associated manipulator with a haptic interface having at least six degrees of freedom being most preferred. Examples of haptic interfaces or masters which can be used with the present invention include the Freedom 6S available from MPB Technologies of Montreal, Canada, and other haptic interfaces commercially available from Sensable Technology of Cambridge, Mass. and MicroDexterity Systems of Albuquerque, N. Mex.

Based on the signals provided by the controller 42, the manipulator 36 executes the desired movement or operation of the tool 34. Thus, any desired dexterity enhancement can be achieved by setting up the controller 42 to perform the appropriate adjustments to the signals sent from the haptic interface 38. For example, this can be accomplished by providing the controller 42 with software which performs a desired dexterity enhancement algorithm. Software dexterity enhancement algorithms can include position scaling (typically downscaling), force scaling (up-scaling for bone and cartilage, downscaling for soft tissue), tremor filtering, gravity compensation, programmable position boundaries, motion compensation for tissue that is moving, velocity limits (e.g., preventing rapid movement into brain, nerve or spinal cord tissue after drilling through bone), and, as discussed in greater detail below, image referencing. These and other examples of possible algorithms are well known in the field of robotics and described in detail in published literature. The ZMP SynqNet® Series Motion Controllers which employ the SynqNet system and are available from Motion Engineering of Santa Barbara, Calif. are one example of a suitable controller for use with the present invention (see www.synqnet.org and www.motioneng.com). Another example of a suitable controller is the Turbo PMAC available from Delta Tau Data Systems of Northridge, Calif.

The robotic surgical system could further have an associated intra-operative positioning sensing or navigation system 44. The navigation system 44 can be configured to monitor not only the position of the tool 34 but also the position of the bone in which the acetabular socket component 14 is to be implanted (e.g., the pelvis) during the joint replacement procedure. The navigation system 44 also can also be configured to monitor the position of the femur during the procedure including during procedures in which a ball portion is being installed on the femur. The position information regarding the tool 34, the femur 16 and the pelvis 18 generated by the navigation system 44 can be communicated back to the controller 42 such that the “real time” position of the tool and the relevant bones can be taken into account in whatever control algorithms are being executed by the controller. Moreover, the navigation system 44 and controller 42 can be configured so to be able to predict the final position of the femur after it is engaged with the acetabular socket component.

With an intra-operative navigation system 44, one of the first steps of the joint replacement procedure can be determining the desired position for the acetabular socket component 14 in the pelvis 18 and then inputting information concerning that desired position into the navigation system 44 and/or controller 42. This can also be done for the ball portion 12 if one is to be installed. This information can then be used by the controller 42 and navigation system 44 to help direct operation of the manipulator 36 during the procedure. Moreover, during the procedure the navigation system 44 and controller 42 can constantly monitor the position of the tool 34 and bones 16, 18 relative to the initial desired position for the replacement joint components and adjust as necessary the control algorithms or provide any necessary warning signals. Those skilled in the art will appreciate that any three dimensional, six degree of freedom position tracking or navigation technology can be used for the navigation system such as optical triangulation or electromagnetic tracking. Such systems are well known in the field of neuro, spine and other types of surgery.

The intraoperative navigation system 44 can further include an image guidance system 46 so that as replacement procedure is performed the position of the tool 34 can be rendered against a preoperative image (e.g., magnetic resonance, computerized tomography, ultrasound or x-ray). If desired, during the procedure, the image data against which the position of the tool is rendered can be updated to provide real time image data using, for example, CT, MR or the like. A combined image guidance and position tracking system is the StealthStation® system available from Medtronic of Minneapolis, Minn.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of installing a replacement joint in a human body:
forming a receptacle having an engagement surface that has an aspherical configuration in a first bone; and
inserting a socket portion of the replacement joint in the receptacle, the socket portion having an engagement surface with an aspherical configuration complementary to the engagement surface of the receptacle in the first bone, the configurations of the engagement surfaces of the socket portion and the receptacle in the first bone being such that inserting the socket portion in the receptacle in the first bone provides the socket portion with at least four degrees of constraint.

2. The method of claim 1 further including the step of supporting a bone cutting medical tool with a manipulator capable of moving the tool with at least one degree of freedom.

3. The method of claim 2 wherein the step of forming the receptacle in the first bone is accomplished by using the manipulator to move the medical tool relative to the second bone.

4. The method of claim 3 further including the step of inputting movement commands for the manipulator through movement of an input device of a haptic interface.

5. The method of claim 2 further including the step of determining a desired position for the socket portion before forming the receptacle in the first bone.

6. The method of claim 5 further including the step of inputting information concerning the desired position into an intra-operative surgical navigation system.

7. The method of claim 1 wherein the step of forming the receptacle in the first bone comprises cutting a plurality of cavities in the second bone with each cavity having an engagement surface with an aspherical configuration.

8. The method of claim 7 wherein the step of inserting the socket portion in the receptacle in the first bone comprises inserting a plurality of socket components each of which is mounted in a respective one of the cavities in the first bone and which has an engagement surface with an aspherical configuration complementary to the engagement surface of its respective cavity in the first bone such that inserting each socket component in its respective cavity provides that socket component with at least four degrees of constraint.

9. The method of claim 8 wherein each of the plurality of socket components has a wedge-shape and each of the respective cavities in the first bone has a complementary wedge shape.

10. The method of claim 1 further including the step of securing the socket portion in the receptacle in the first bone.

11. The method of claim 10 wherein the socket portion is secured in the receptacle in the first bone using cement.

12. The method of claim 10 wherein the socket portion is secured in the receptacle in the first bone using an interlocking assembly.

13. The method of claim 12 wherein the interlocking assembly includes an undercut edge of the receptacle, a tapered edge of the socket portion and a wedge screw.

14. The method of claim 1 further including the step of mounting a ball portion of the replacement joint which is received in the socket portion on an end of a first bone.

15. The method of claim 6 further including the step of monitoring the position of the first bone using the intra-operative surgical navigation system while forming the receptacle in the second bone.

16. The method of claim 15 further including the step of communicating information concerning the position of the first bone to a controller for the manipulator.

17. The method of claim 16 further including the step of monitoring the position of the second bone using the intra-operative surgical navigation system while forming the receptacle in the first bone.

18. A medical implant for replacing a joint in a human body:
a ball portion; and
a socket portion for receiving the ball portion, the socket portion having an engagement surface with an aspherical configuration that provides at least four degrees of constraint when the socket portion is inserted in a receptacle formed in a bone that has a complementary aspherical configuration.

19. The medical implant of claim 17 wherein the socket portion comprises a plurality of socket components each of which has an engagement surface with an aspherical configuration that provides at least four degrees of constraint when the socket portion is inserted in a respective cavity of the receptacle in the bone having a complementary aspherical configuration.

20. The medical implant of claim 19 wherein each of the plurality of socket components has a wedge shape.

21. The medical implant of claim 19 wherein the socket portion has an edge that tapers inward as it extends from an upper surface of the socket portion towards a lower surface of the socket portion.

22. The medical implant of claim 21 further including a wedge screw for engaging the socket portion and a bone.
